(19) United States
(12) Patent Application Publication
(10) Pub. No.: US 2021/0247291 A1
(43) Pub. Date: Aug. 12, 2021

(54) PROBING MECHANICAL PROPERTIES OF BIOLOGICAL MATTER

(71) Applicant: LUMICKS TECHNOLOGIES B.V., Amsterdam (NL)

(72) Inventors: Gijs Jan Lodewijk WUITE, Amsterdam (NL); Raya SORKIN, Amsterdam (NL); Douwe KAMSMA, Amsterdam (NL); Gerrit SITTERS, Amsterdam (NL); Erwin Johannes Gerard PETERMAN, Amsterdam (NL); Giulia BERGAMASCHI, Amsterdam (NL); Mattijs DE GROOT, Amsterdam (NL)

(73) Assignee: LUMICKS TECHNOLOGIES B.V., Amsterdam (NL)

(21) Appl. No.: 17/052,061

(22) PCT Filed: May 2, 2019
(86) PCT No.: PCT/NL2019/050262

§ 371 Date: Oct. 30, 2020

(30) Foreign Application Priority Data
May 2, 2018 (NL) 2020862

(51) Int. Cl. G01N 15/14; B01L 3/00

(52) U.S. Cl. CPC G01N 15/14; G01N 2015/0065; B01L 2400/0436; B01L 3/508

(57) ABSTRACT

A method for probing mechanical properties of cellular bodies includes: providing a plurality of particles in a fluid medium contained in a holding space of a sample holder, each of the plurality of particle being attached to a cellular body; generating a resonant bulk acoustic wave in the holding space, the resonant bulk acoustic wave exerting an acoustic force on each of the plurality of particles, each of the plurality of particles having an acoustic contrast factor and a size, the acoustic contrast factor and the size being selected such that the force exerted on a particle is larger than the force exerted on the cellular body to which the particle is attached; measuring a displacement of a particle in response to the exertion of the force on the particle, the measured displacement being associated with a mechanical property of the cellular body attached to the particle.

### TECHNICAL FIELD

The present disclosure relates to probing mechanical properties of biological matter, and, in particular, though not exclusively, to methods and systems for probing mechanical properties of biological matter using acoustic waves, and a computer program product for executing such methods.

### BACKGROUND OF THE INVENTION

The measurement of mechanics of biological matter, e.g. cellular bodies and/or biological soft matter layers such as a tissue layers, lipid bilayers, organ on chip, etc., is crucial for a better understanding of biological processes, e.g. cellular responses during the progression of many diseases, including malaria, anaemia and cancer. A variety of biophysical techniques, including atomic force microscopy, optical tweezers, and micro-pipette aspiration, have been developed to spatially manipulate a cell in order to probe its mechanics.

Hwang et a., describe in their article Cell Membrane Deformation Induced by a Fibronectin-Coated Polystyrene Microbead in a 200-MHz Acoustic Trap, IEEE transactions on ultraconics, ferroelectrics and frequency control, vol. 61, No. 3, March 2014 pp. 399-406, a tool for probing mechanics of a single cell using an acoustic trap. The acoustic trap is used to trap a microbead (a micron-sized spherical particle), transport the microbead to a cell and attach trapped microbead to the membrane of the cell. By moving the trap laterally, a lateral force can be applied to the cell membrane which induces a stretching of the cell.

Although cell mechanics can be probed, this technique has the disadvantage that it entails a single cell manipulation tool requiring an acoustic trap with a small focus (small enough to manipulate a microbead) which can be moved around. Further, the tool allows application of a force in the transverse direction, i.e. a direction parallel to the surface to which the cell is attached, so that the deformation of the cell may be influenced by the way the membrane interacts with the surface, e.g. by drag forces.

More in general, current state of the art acoustic probing techniques are single cell probing techniques which are not suitable for simultaneously determining individual cell mechanics parameters of a large number of cellular bodies and/or for different parts of a biological soft matter layer.

Additionally, many of these techniques suffer from low signal-to-noise ratio, slow detection, small sample size and/or only localised and indirect interaction with the cellular membranes of probed cells.

WO2014/200341 describes a manipulation system for investigating molecules, having a sample holder constructed to hold a sample comprising a plurality of molecules, e.g. DNA, attached on one side to a surface in the sample holder and on another side attached to a microbead of a plurality of microbeads. The system includes an acoustic wave generator to generate a resonant bulk acoustic wave in the sample holder, which exerts a force on the microbeads. An optical detector is configured to track the displacement of the microbeads in three dimensions one of which is perpendicular to the surface of the sample holder (the z-direction) when an acoustic force is applied. The displacement of the microbeads in response to the applied force can be correlated with properties of the molecule.

When trying to apply this technique to study mechanical properties of biological matter, such as cellular bodies and/or biological soft matter layers problems arise. The dimensions of cellular bodies or a biological soft matter layer are substantially larger than the dimensions of molecules. Thus, unlike molecules, cellular bodies or a biological soft matter layer will experience an acoustic force when being placed in a bulk acoustic field. Hence, the acoustic force field need to be selected such that a microbead experiences a substantial force while the force on the cellular body or soft matter layer should be small. When using conventional microbeads, such as polysterene or glass microbeads, the size of the microbead need to be in the order of size of the cell to exert a sufficient force. Large microbeads may have a large contact area and thus do not allow to controllably probe the mechanical response of specific parts of a cellular body or soft matter layer. However, the force on the microbeads scales with the third power of the volume of the microbeads so possibilities to reduce the size of conventional microbeads will be limited.

Further, when studying single molecules using acoustic force spectroscopy as described in WO2014/200341, the microbeads are optically tracked against an optically homogeneous background as the molecules are substantially smaller than the microbeads. In contrast, when examining cellular bodies or soft matter layers, the microbeads need to be optically tracked against a background of the cellular bodies or structures in the soft matter layer that have dimensions of the same dimensions of the microbeads or larger. Additionally, when trying to track microbeads in the z-direction, the image of the cellular body or soft matter layer may interfere with the optical signal of the microbead, thereby deteriorating the accuracy of the measurements.

Hence, from the above it follows that there is a need in the art for methods and systems that enable accurate high throughput (multiplexed) probing of mechanics of cellular bodies or biological soft matter layers. In particular, there is a need in the art for accurate high throughput probing of mechanics of cellular bodies or soft matter layers which allows simultaneous determination of mechanics parameters of a large number of cellular bodies or different parts of a biological soft matter layer so that large data sets for statistical analysis can be efficiently acquired.

### SUMMARY OF THE INVENTION

It is an objective of the invention to reduce or eliminate at least one of the drawbacks known in the prior art. It is an aim of the invention to enable multiplexed mechanical probing of mechanics of biological matter such as cellular bodies and/or biological soft matter layers using an acoustic wave technique. In an aspect, the invention may relate to a method of probing mechanical properties of biological matter, e.g. cellular bodies and/or biological soft matter layers, including: providing a plurality of particles, preferably particles or nanoparticles, in a fluid medium contained in a holding space of a sample holder, each of the plurality of particles being attached to a cellular body or a part of a biological soft matter layer positioned on a surface of the holding space; generating a resonant bulk acoustic wave in the holding space, the resonant bulk acoustic wave exerting an acoustic force on each of the plurality of particles in a direction away from the surface of the holding space or in a direction towards the surface of the holding space, each of the plurality of particles having an acoustic contrast factor and a size, the acoustic contrast factor and the size being selected such that the force exerted on a particle is larger than the force exerted on the cellular body or the part of the biological soft matter layer to which the particle is attached; for at least part of the particles attached to a cellular body or part of a biological soft matter layer, measuring a displacement of a particle in response to the exertion of the force on the particle, the measured displacement being associated with at least one mechanical property of the cellular body or part of a biological soft matter layer.

In another aspect, the invention may relate to a method for probing mechanical properties of cellular bodies, wherein the method may include: providing a plurality of particles, preferably microparticles or nanoparticles, and cellular bodies in a fluid medium contained in a holding space of a sample holder and fixating the cellular bodies to a surface of the holding space, wherein each of the plurality of particles is attached to one of the cellular bodies; generating a resonant bulk acoustic wave in the holding space, the resonant bulk acoustic wave exerting an acoustic force on each of the plurality of particles in a direction away from the surface of the holding space or in a direction towards the surface of the holding space, each of the plurality of particles having an acoustic contrast factor and a size, the acoustic contrast factor and the size being selected such that the force exerted on a particle is larger than the force exerted on the cellular body to which the particle is attached; and, for at least part of the particles which attached to a cellular body, measuring one or more displacements of a particle in response to the exertion of the force on the particle, the measured one or more displacements being associated with at least one mechanical property of the cellular body.

In an embodiment, the displacements may be measured using an optical detector, e.g. an optical tracking system, for tracking one or more of the particles in one or more directions (e.g. in the x-y plane parallel to the surface on which the cells are fixated or in the z-axis normal to the surface on which the cells are fixated).

The invention entails acoustic force spectrometry methods and systems enabling multiplexed non-invasive mechanical measurements on cellular bodies or biological soft matter layers, such that mechanics of such biological matter can be probed. Here, cellular bodies may be whole cells such as immune cells, tumor cells, blood cells, muscle cells, etc. However, the cellular bodies may also be cell portions like subcellular organelles, cell nuclei, and/or mitochondria or, the cellular bodies may also be unicellular or pluricellular, such as small clumped cell groups, plant or animal biopts, dividing cells, budding yeast cells, colonial protists, etc. The cellular bodies may also be animal embryos in an early stage of development (e.g. the morula-stadium of a mammal, possibly a human embryo). In particular cases different types of cellular bodies may be studied together. E.g., cellular bodies from a mucosal swab, blood sample, or other probing techniques could be used. Similarly, a biological soft matter layer may include, e.g. a tissue layer, lipid bilayer, organ on chip, etc. These layers may or may not include cellular bodies.

In order to probe the mechanics, particles are attached to cellular bodies or a biological soft matter layer, which may be positioned on the surface of a sample holder. Part of the sample holder may be configured as an acoustic chamber in which an acoustic standing wave can be generated. The particles may be functionalized for adhesion to the cellular bodies and/or the biological soft matter layer. Further, the particles are selected to have an acoustic contrast factor such that the force exerted on a particle is larger than the force exerted on the cellular body or part of the biological soft matter layer to which the particle is attached. This way, the acoustic force field may, via the particle, pull or push on the cells or the layer that are (is) positioned on the surface of a flow cell in a direction that is normal to the surface, i.e. the z-direction. An optical tracking technique may be used to monitor the displacement of the particles in 3 dimensions (including the x-y plane parallel to the surface of the sample holder and the z-direction perpendicular to the surface of the sample holder) as a function of the force applied to the particles.

A particle of a predetermined size and material may be selected to provide a sufficiently high acoustic contrast relative to the cells in order to ensure that the acoustic force applied to the cell is negligible, while the acoustic force applied to the particle is sufficient for probing mechanical properties of the cell. The acoustic contrast factor is a way to quantify how strong a certain material interacts with an acoustic field in a specific fluid medium. The acoustic contrast factor, together with the size of the particle determines the force experienced by the particle in a given (fluid) medium due to an acoustic field. The acoustic contrast factor depends on the difference in density and speed of sound of the material used compared to the medium. Additionally, a particle of a predetermined size and material may be selected to provide a sufficiently high optical contrast with the cell background so that displacements of particles due to the acoustic force can be accurately tracked.

In an embodiment, the particles may include hollow particles, which may be filled with a gas, a gas mixture (including air) or a liquid; preferably the hollow particles including hollow inorganic particles (e.g. oxide-based, for example silicon oxide-based hollow particles, glass-based hollow particles or ceramic-based hollow particles) and/or the hollow particles including hollow organic particles (e.g. polymer-based hollow particles, for example polyvinyl alcohol (PVA) based hollow particles).

In an embodiment, the particles may have an acoustic contrast factor (in a water-based fluid medium) that is larger than 0.5 or smaller than −0.5. In another embodiment, the particles may have an acoustic contrast factor (in a water-based fluid medium) that is larger than 0.6 or smaller than −0.6.

In an embodiment, the hollow particles may have a low compressibility (e.g. a ‘hard’ shell material). To that end, in an embodiment, the hollow particles may have a shell of a material having a Young's modulus selected between 1 and 1000 GPa. In various embodiments, the shell material may have a Young's modulus between 50-90 GPa (e.g. a glass shell material) or a Young's modulus between 1-10 GPa (e.g. a polymer based shell material such as polyvinyl alcohol) or a Young's modulus of more than 100 GPa (e.g. a ceramic based shell material).

In an embodiment, the hollow particles may have a shell thickness up to 5 micron, preferably between 0.1 and 5 micron.

In an embodiment, the particle material may have an optical refractive index (real part in the visible domain) that differs at least 80% with the refractive index of the medium (e.g. diamond having a refractive index of 2.4-2.5 in the visible range). In another embodiment, the particle material may have an optical refractive index (real part in the visible domain) that differs at least 20-25%, with the refractive index of the medium (e.g. air-filled hollow particles has a refractive index of 1 in the visible range).

In an embodiment, the size of the particles may be selected to be 20% of the size of the cellular bodies or smaller. In an embodiment, the (average) size of the particle may be selected between 0.2 and 20 micron and wherein the size of the cellular bodies is selected between 2 and 100 micron.

In an embodiment, the flow cell may be configured to have a first acoustic resonant frequency wherein the force exerted on a particle is in a direction away from the surface of the holding space of the sample holder and at a second acoustic resonant frequency wherein the force exerted on a particle is in a direction towards the surface of the holding space of the sample holder.

Hence, the acoustic chamber may be configured so that at least two acoustic resonant modes can be generated in the acoustic chamber. Here, a first resonant mode may exert a pulling force on a particle and a second resonant mode may exert a pushing force on the particle. When pulling on a cellular body with a bead, a pulling force will be exerted on the lipid membrane of the cellular body. In an embodiment, this may eventually result in pulling out a membrane tether. The response of the pulling action, e.g. the displacement of the particle, will provide information about the membrane and its connection to the cytoskeleton of the cell. Alternatively, when the particle pushes a cellular body, a pushing force will be exerted on the cytoskeleton of the cellular body. This way information about the cytoskeleton may be obtained (similar to an AFM experiment).

In an embodiment, a portion of the particles may be functionalized using one or more primers comprising one or more interaction moieties for adhesion to at least part of the cellular body, preferably an interaction moiety including at least one of: antibodies, viruses, viral particles, peptides, biological tissue factors, biological tissue portions, antigens, proteins, ligands, lipid (bi)layers, fibronectin, cellulose, nucleic acids, RNA, small molecules, allosteric modulators, (bacterial) biofilms, and specific atomic or molecular surface portions (e.g. a gold surface).

In an embodiment, hollow particles polyvinyl alcohol (PVA) particles may be functionalized using antibodies in order to adhere the hollow particles to certain cell types, such as cancer cells.

The invention allows detecting changes in cellular mechanics of cells, e.g. Red Blood Cells (RBCs) or changes in the mechanics of a biological soft matter layer. For example, a force response can be determined for healthy cells and diseased cells. Based on the differences in the force response a cell can be classified in a healthy cell or a diseased cell. Further the force response can be determined before treatment, during and after treatment with a particular drug, e.g. Cytochalasin D (drug disrupting actin filaments in the cytoskeleton). Here, a treatment may refer to an in vitro treatment of cells with one or multiple drugs, or, alternatively, a process wherein cells are sampled from a patient undergoing treatment.

The mechanical response of cellular bodies, such as RBCs, may be detected by following the displacement of the particles. A viscoelastic model may be used to determine force-extension curves, which can be used to classify cells, e.g. classify healthy and treated RBCs, where the latter presented larger extension length and a broader distribution of relaxation times. Additionally, the invention allows detecting changes in the mechanical parameters when cells were treated with a specific compound, e.g. cross linkers, such as formaldehyde.

In an embodiment, the method may further comprise: inserting a plurality of cellular bodies and a plurality of particles in the medium of the holding space of the sample holder. In an embodiment, a particle is attached to a cellular body. In another embodiment, the particles and the cellular bodies provided separately into to holding space and mixed in order to allow at least part of the particles to attach to a least part of the cellular bodies; controlling a resonant bulk acoustic wave in the sample holder in order to exert a force to the particles, the acoustic force being selected to be smaller than a gravitational force that pulls cellular bodies towards the surface of the holding space. The gravitational force pulling the cellular bodies towards the surface of the holding space, wherein the acoustic force acting on a particle attached to a cellular body ensures that the cellular body will land onto the surface with the particle on top.

In an embodiment, the method may further comprise: inserting a plurality of cellular bodies and a plurality of particles in the medium of the holding space of the sample holder, wherein (at least part of) the particles have a density lower than the density of the medium, preferably (at least of) the particles including hollow air-filled or gas-filled particles. In an embodiment, a particle is attached to a cellular body. In another embodiment, the particles and the cellular bodies provided separately into to holding space and mixed in order to allow at least part of the particles to attach to a least part of the cellular bodies; a gravitational force pulling the cellular bodies towards the surface of the holding space, wherein the buoyancy force of a particle attached to a cellular body ensures that the cellular body will land onto the surface with the particle on top.

In accordance with the presented method embodiments, an embodiment of the acoustic fore spectrometry system comprises a sample holder comprising a holding space for holding a sample providing a plurality of particles, preferably micro particles or nanoparticles, in a fluid medium contained in a holding space of a sample holder, each of the plurality of particles being attached to a cellular body and each cellular body being positioned on a surface of the holding space and an acoustic wave generator connectable or connected with the sample holder to generate an acoustic wave in the holding space exerting a force on the sample.

Typically, for the fluid medium, water or a water-based buffer is used. However also oils and (hydro)gels could be used. In case of a sample comprising patient material, the fluid could be a bodily fluid. The sample holder may comprise a wall providing the holding space with a functionalised surface portion to be contacted, in use, by at least part of the sample.

The system, in particular the sample holder, may be designed in such a way that an acoustic amplitude maximum or minimum is located at or near the functionalised surface of the sample holder.

The sample holder may comprise a recess forming the holding space. The sample holder may be unitary or constructed from plural objects, e.g. a part that is at least locally U-shaped in cross section and a cover part to cover and close the U-shaped part providing an enclosed holding space in cross section. The enclosed holding space may be enclosed in all directions, or it may have one or more entrance and/or exit ports, e.g. forming a continuous channel.

The acoustic wave generator may be permanently attached to the sample holder, possibly integrated in a portion of the sample holder. In another embodiment the acoustic wave generator may be repeatedly attachable to the sample holder, or be connected to form an acoustic cavity with the sample holder via an acoustic transmitting medium. As examples, ultrasound waves may be generated by piezoelectric generators, electromechanical generators, optical generators (e.g. subjecting a portion to a series of laser pulses), and other techniques, possibly in combination. For creation of a standing wave the sample holder including the sample fluid and possibly structures attached to the sample holder may form an ultrasound cavity for particular frequencies, in one or more directions. The sample holder may then be designed to prevent or to promote mode-mixing of different and/or differently oriented acoustic modes.

The acoustic wave generator may comprise or be a bulk acoustic wave generator to generate a bulk acoustic wave in the holding space and/or in a sample contained therein.

In an embodiment, a portion of the surface of the sample holder may be provided with one or more primers. The primers may comprise one or more types of interaction moieties and/or precursors thereof. In particular the functionalised surface portion may include at least one of viruses or viral particles, antibodies, peptides, biological tissue factors, biological tissue portions, bacteria, antigens, proteins, ligands, cells, tissues, viruses, (synthetic) drug compounds, lipid (bi)layers, fibronectin, cellulose, nucleic acids, RNA, small molecules, allosteric modulators, (bacterial) biofilms, “organ-on-a-chip”, specific atomic or molecular surface portions (e.g. a gold surface) to which at least part of the sample tends to adhere with preference relative to other surface portions, nanostructured or micro-structured surface portions e.g. micropillars, microridges, etc.

By providing the functionalised surface portion with one or more primers, particular predetermined interactions between the cell(s) of the cellular body and the functionalised surface portion may be influenced or caused on contact of the cellular body with such portion. E.g. a cellular body may adhere to the wall.

In an embodiment, the sample holder is connected or connectable to a flow system for introducing a fluid into the holding space and/or removing a fluid from the holding space, e.g. for flowing fluid through the holding space. The fluid flow system may be comprised in the manipulation system. The fluid flow system may comprise one or more of reservoirs, pumps, valves, and conduits for introducing and/or removing one or more fluids, sequentially and/or simultaneously.

Fluid that thus is introduced into, removed from or flowed through the holding space may comprise sample material, e.g. one or more of sample fluids, cellular bodies, actors on the cellular bodies, etc. Thus, one or more parts of a sample may be recovered after an experiment and/or different sample conditions may be provided, e.g. one or more of different pH-values, different dilutions such as salt concentrations, different fluid compositions, and different cellular bodies sequentially or in parallel. Also, actors on cellular bodies may be introduced, e.g. nutrients, chemical substances, viruses, etc. The fluid may also comprise one or more dissolved or, in particular in hydrogel and/or aerogel-based fluids, entrained gases, like N₂, CO₂, O₂, noble gases, like Ne, Ar, noxious gases like CO, O₃, NOₓ, and other gases that may have a biological effect such as cyanide-containing gases, ethylene, etc. The gas may also be a gas mixture of different components, with a controlled composition or with an uncontrolled composition like ambient air. It is considered that acoustic index variations which may disturb a desired acoustic force profile within the holding space, such as density variations in the fluid, in particular gas bubbles of a size in the order of an interior dimension of the holding space such as spanning a significant portion of a width and/or height of the holding space, should be prevented, at least during study of the sample.

Control over the amount and/or composition of the fluid within the sample holder may also be used to control a sample volume.

The system may be configured to introduce the fluid into the holding space and/or remove the fluid from the holding space, e.g. flowing the fluid through the holding space, simultaneously with operation of the acoustic wave generator to generate an acoustic wave in the holding space exerting a force on the sample. Thus, a motion dynamic component, e.g. flow-induced lateral force component may be included in the study.

The system may thus also be included in a flow cytometry system or other flow system, e.g. integration with an on-line flushing system.

The flow direction may be perpendicular to the direction of the acoustic force, or at least a main force direction component from that.

In an embodiment, the acoustic wave generator is controllable to adjust at least one of frequency and amplitude for generating adjustable acoustic waves, preferably time-dependent, wherein in particular the acoustic wave may be a standing wave.

This enables parameterised studies. In a particular case, the amplitude may be adjusted over at least two orders of magnitude. Acoustic frequencies may depend on the geometry of the sample holder but they generally may lie between 1 and 100 MHz A sample holding space open size in any one of three perpendicular directions, e.g. height, width, length of the holding space may be in a range between 1 and 1000 micrometres, so that the holding space may typically have a volume in a range of 0.1 microliter-100 microliters, or even up to a millilitre, with a large variety of geometries, like “lab on a chip” sample holders. Important is that the sample holder can provide and sustain the acoustic wave effectively in the sample.

The mechanical response of a single cellular body or a plurality of cellular bodies may be measured simultaneously. A large plurality of cellular bodies may form a surface bulk, e.g. a bacterial colony. In an embodiment, plural individual cellular bodies may be manipulated and/or measured separately but in parallel, as opposite to surface bulk studies.

The acoustic wave signal may vary over several orders of magnitude, e.g. lasting from sub-second to tens of minutes or even longer, depending on the nature and robustness of the sample and processes in it.

A suitable frequency of the acoustic wave may be determined by the dimensions of at least a portion of the sample holder (either or not in combination with the acoustic generator), e.g. the acoustic cavity; the frequency may be a resonance frequency of the acoustic wave in that portion, possibly being associated with a standing acoustic wave in that portion. An optimum frequency may be determined beforehand and/or in operation. It is considered that a resonance frequency may exert an acoustic force of several orders of magnitude higher than a non-resonant acoustic wave.

A time dependent adjustment of the acoustic force may enable studies based on predetermined frequency and/or amplitude patterns, e.g. pulses, ramps, repetitive modulations and/or sweeps such as frequency chirps. Different frequency spectra may be applied as well. Using such techniques particles may be manipulated selectively or collectively, e.g. by pushing or pulling.

The system may comprise plural acoustic wave generators, which may be arranged for generating acoustic waves which mutually differ with respect to at least one of frequency, amplitude, and a time dependency of the respective frequency and/or amplitude and/or of which at least one is controllable to adjust a frequency and/or an amplitude of the respective acoustic wave for generating adjustable acoustic waves, preferably time-dependent. One or more further acoustic wave generators may thus be provided for generation of complex force fields, in a particular embodiment acoustic wave generators are connected with the sample holder to generate acoustic waves in the holding space from perpendicular directions, which may be separately controllable.

According to an embodiment the acoustic force spectroscopy system may comprise a detector for detecting a response of one or more particles attached to one or more cellular bodies respectively to (a force exerted by) the acoustic wave on the particles. Mechanical properties of the one or more cellular bodies may therewith be investigated.

The detector may comprise an optical detector. The optical detector may comprise a photodiode, an array of photodiodes, a camera and/or a microscope, but for particular experiments a photocell without image resolution could also suffice, e.g. for bulk measurements. Digital photo and/or film cameras are considered suitable, preferably having an adjustable imaging frame rate. The detector may be wavelength selective, e.g. comprising one or more colour filters. Possibly plural detectors are provided which are wavelength selective for different wavelengths.

In a particular embodiment, the detector comprises a confocal microscope and/or superresolution microscope, e.g. a (near field) scanning optical microscope (“SOM”/“NSOM”), structured illumination microscope (“SIM”), multicolour excitation light sources.

Typically, bright field illumination may be used, e.g. LED-illuminated. However, dark field imaging may also be employed. Using software (portions of) the sample may be studied with an accuracy below the diffraction limit, even if the microscope itself is not a superresolution microscope. The detector does however not have to be a microscope. Other options include surface plasmon resonance detection and/or lensless imaging (e.g. imaging without lenses by placing a camera chip directly under the sample).

Further, acoustic detection may be employed; Surface acoustic waves enable detection of objects on the surface, e.g. by effects on the wave, like differences in amplitude, phase and/or direction of propagation due to absorption and/or reflection of acoustic energy etc. A suitable acoustic detector comprises a piezo actuator serving as sensor element.

Although contact detection and/or other forms of detection may be used in combination with acoustic force studies, optical detection may be preferred since this may be done with little to no effect on or interaction with a biological cellular body. Further, many different proven optical techniques and systems are available. For optical detection, the system may be provided with a light source. The light source may be polychromatic or monochromatic, which may cause or rather preclude optical interaction with (portions of) the sample and which may reduce or prevent optical (chromatic) aberrations for optical detection. Optical detection relying on any of optical interference, refraction, diffraction, may benefit from a light source providing illumination with plane wave fronts or otherwise controlled wave fronts at the location of the sample.

In embodiments, the system comprises one or more of a light source, a memory, a tracking system for tracking one or more of the particles and a controller for performing microscopy calculations and/or analysis associated with the microscopy detection technique. The tracking system may be configured to perform 2-dimensional (“2D”) and/or 3-dimensional (“3D”) tracking. In an embodiment, 3D tracking can be used to determine a speed of a particle through a surrounding medium, which may also be used to determine an acoustic force of the system on the particle and/or interaction of the particle with an acoustic force of the system. The tracking system and/or the controller may also be connected with the acoustic wave generator to control operation of the system. In some embodiments, besides tracking the particles the cellular bodies may also be tracked in order to obtain information on the cellular bodies.

The system may comprise a sensor and a controller connected or connectable with the acoustic wave generator for controlling operation of the acoustic wave generator in response to a signal from the sensor. Thus, a feedback system may be provided.

In an embodiment, the system comprises for manipulating one or more of the particles or cellular bodies one or more of an optical tweezers system, a magnetic tweezers system, an electrostatic tweezers system and contact probes, wherein one or more of the particles or cellular bodies may be accordingly prepared and a preparation system for that may be provided.

In an embodiment, the system comprises a light source for excitation and/or interrogation of one or more optically active portions in the sample, e.g. chromophores, etc., e.g. for using fluorescence detection. The light source may comprise a laser, the light source preferably is wavelength tuneable.

The system may be provided as a stand-alone system, as a worktop-instrument or even as a hand-held instrument.

An embodiment further comprises a detector to produce a digital image of a focal plane and wherein the system is provided with a calculation device to calculate a position of one or more of the particles and/or cellular bodies in a direction perpendicular to the focal plane by processing of an interference pattern caused by one or more of the cellular bodies which are not in focus. This enables interference detection and determining and tracking particles and/or cellular bodies in a direction along an imaging direction, perpendicular to a direction of the acoustic wave and/or the functionalised surface. An embodiment may comprise a thermal element for adjusting a temperature and/or a temperature profile of the sample holder. The thermal element may comprise a simple electrical heater wire or a more intricate element like one or more Peltier elements which may enable precise thermal control and which may provide both heating and cooling.

In a further aspect a sample holder for the system and/or method described herein are provided. The sample holder comprises a holding space for holding a sample comprising one or more particles and cellular bodies in a fluid medium, and an acoustic wave generator connected with the sample holder to generate an acoustic wave in the holding space exerting a force on the sample, wherein the sample holder comprises a wall providing the holding space with a functionalised wall surface portion to be contacted, in use, by at least part of the cellular bodies and/or biological soft matter layer.

The sample holder may be formed with a connecting portion to be connected with a corresponding counter connector of a device comprising associated ultrasound power generator and detection system, e.g. one or more of light sources and optical detectors, for provision of the system and/or performance of the method.

The method and system each combine aspects of acoustic force spectroscopy, microfluidics, surface functionalization and live (super-resolution) video tracking of plural particles and/or cellular bodies, possibly even single particle live super-resolution video tracking, to perform force-spectroscopy on cellular bodies down to the single particle level in a highly parallelized fashion. Generating resonant bulk acoustic waves within a microfluidic cavity allows to directly and instantly apply forces on the particles.

In combination with live (single-particle) video tracking the present techniques enables to precisely detect mechanical properties of biological matter such as cellular bodies and/or biological soft matter layers. Note that motion-detection may not require accurate position detection at any given time, and position determination of a sample portion at a particular time need not require detection and/or tracking of movement of the sample portion at that time.

The invention is particularly suited for multiplexed measurements of mechanics of biological matter, e.g. cellular bodies and/or biological soft matter layers, which allows fast acquisition of large statistics. Because of the easy and straightforward implementation, it can potentially be used for biomedical/diagnostic applications.

As will be appreciated by one skilled in the art, aspects of the present invention may be embodied as a system, a method or a computer program product. Accordingly, aspects of the present invention may take the form of an entirely hardware embodiment, an entirely software embodiment (including firmware, resident software, micro-code, etc.) or an embodiment combining software and hardware aspects that may all generally be referred to herein as a “circuit,” “module” or “system.” Functions described in this disclosure may be implemented as an algorithm executed by a processor/microprocessor of a computer. Furthermore, aspects of the present invention may take the form of a computer program product embodied in one or more computer readable medium(s) having computer readable program code embodied, e.g., stored, thereon.

Any combination of one or more computer readable medium(s) may be utilized. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples of a computer readable storage medium may include, but are not limited to, the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of the present invention, a computer readable storage medium may be any tangible medium that can contain, or store, a program for use by or in connection with an instruction execution system, apparatus, or device.

A computer readable signal medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium may be transmitted using any appropriate medium, including but not limited to wireless, wireline, optical fiber, cable, RF, etc., or any suitable combination of the foregoing. Computer program code for carrying out operations for aspects of the present invention may be written in any combination of one or more programming languages, including an object oriented programming language such as Java™, Smalltalk, C++ or the like and conventional procedural programming languages, such as the “C” programming language or similar programming languages. The program code may execute entirely on the user's computer, partly on the user's computer, as a stand-alone software package, partly on the user's computer and partly on a remote computer, or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Aspects of the present invention are described below with reference to flowchart illustrations and/or block diagrams of methods, apparatus (systems), and computer program products according to embodiments of the present invention. It will be understood that each block of the flowchart illustrations and/or block diagrams, and combinations of blocks in the flowchart illustrations and/or block diagrams, can be implemented by computer program instructions. These computer program instructions may be provided to a processor, in particular a microprocessor or a central processing unit (CPU), of a general purpose computer, special purpose computer, or other programmable data processing apparatus to produce a machine, such that the instructions, which execute via the processor of the computer, other programmable data processing apparatus, or other devices create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

The flowchart and block diagrams in the figures illustrate the architecture, functionality, and operation of possible implementations of systems, methods and computer program products according to various embodiments of the present invention. In this regard, each block in the flowchart or block diagrams may represent a module, segment, or portion of code, which comprises one or more executable instructions for implementing the specified logical function(s). It should also be noted that, in some alternative implementations, the functions noted in the blocks may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustrations, and combinations of blocks in the block diagrams and/or flowchart illustrations, can be implemented by special purpose hardware-based systems that perform the specified functions or acts, or combinations of special purpose hardware and computer instructions.

Moreover, a computer program for carrying out the methods described herein, as well as a non-transitory computer readable storage-medium storing the computer program are provided. A computer program may, for example, be downloaded (updated) to the existing data processing system or be stored upon manufacturing of these systems.

Elements and aspects discussed for or in relation with a particular embodiment may be suitably combined with elements and aspects of other embodiments, unless explicitly stated otherwise. Embodiments of the present invention will be further illustrated with reference to the attached drawings, which schematically will show embodiments according to the invention. It will be understood that the present invention is not in any way restricted to these specific embodiments.

### BRIEF DESCRIPTION OF THE DRAWINGS

The above-described aspects will hereafter be explained with further details and benefits with reference to the drawings showing a number of embodiments by way of example.

FIG. 1 schematically depicts an acoustic force spectroscopy system according to an embodiment of the invention;

FIGS. 2A and 2B schematically depict details of a sample holder for use in an acoustic force spectroscopy system according to an embodiment of the invention;

FIG. 3A-3F schematically depicts a process for probing mechanical properties of cells according to an embodiment of the invention;

FIG. 4 depicts mechanical responses of cellular bodies measured using an acoustic force spectroscopy system according to an embodiment of the invention;

FIG. 5 depicts experimental data of high acoustic contrast particles according to an embodiment of the invention;

FIG. 6 schematically depicts a process for probing mechanical properties of cells according to another embodiment of the invention;

FIG. 7 schematically depicts part of a flow cell for an acoustic force spectroscopy system according to an embodiment of the invention;

FIG. 8 schematically depicts acoustic resonant modes of part of a flow cell for an acoustic force spectroscopy system according to an embodiment of the invention;

FIG. 9 depicts a process for preparing cells for a mechanical probing process according to an embodiment of the invention;

FIG. 10 schematically depicts a process of classifying cells on the basis of mechanical according to an embodiment of the invention.

### DETAILED DESCRIPTION OF EMBODIMENTS

It is noted that the drawings are schematic, not necessarily to scale and that details that are not required for understanding the present invention may have been omitted. The terms “upward”, “downward”, “below”, “above”, and the like relate to the embodiments as oriented in the drawings, unless otherwise specified. Further, elements that are at least substantially identical or that perform an at least substantially identical function are denoted by the same numeral, where helpful individualised with alphabetic or subscript numeric suffixes.

While the embodiments and examples hereunder are described with reference to cellular bodies, it is appreciated that these embodiments and examples are not limited thereto and also include systems and methods for probing mechanical properties of biological soft matter layers such as a tissue layers, lipid bilayers, organ on chip, etc.

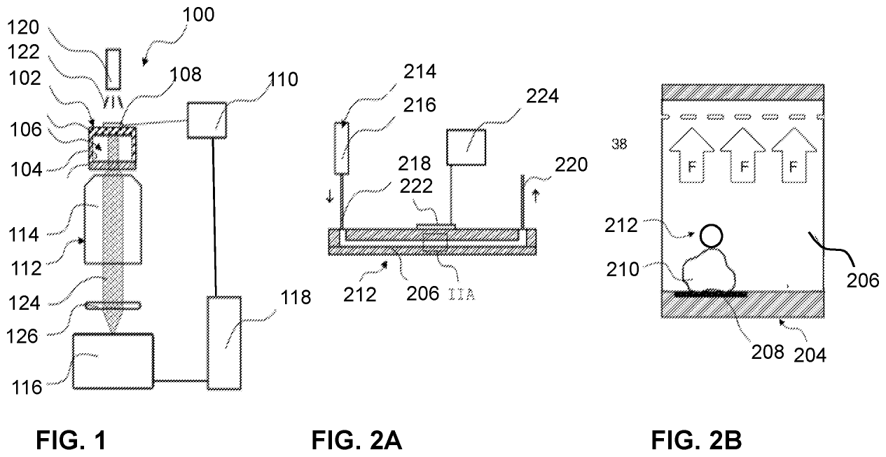

FIG. 1 schematically depicts an acoustic force spectroscopy system according to an embodiment of the invention. FIG. 2A schematically depicts a cross section of a sample holder and FIG. 2B is a detail of the sample holder of FIG. 2A as indicated with “IIA”.

The system 100 comprises a sample holder 102 comprising a holding space 104 for holding a sample 106 comprising one or more biological cellular bodies in a fluid medium wherein each of (at least part of) the cellular bodies is attached to a particle, a microparticle or in some cases a nanoparticle. These particles, microparticles and nanoparticles are hereafter referred to in short as particles. The fluid preferably is a liquid or a gel. The system further comprises an acoustic wave generator 108, e.g. a piezo element, connected with the sample holder 102 to generate an acoustic wave in the holding space exerting a force on the particles in the sample. The acoustic wave generator may be connected to a controller 110.

As shown in FIG. 2B, the sample holder 202 comprising the holding space 206 may comprise a wall 204 comprising a surface for supporting cells. In an embodiment, the surface may be provided with a functionalised surface portion 208 to be contacted, in use, by part of a plurality of samples, each sample including a cellular body 210 attached to a particle 212. Hence, on side of the cellular body is connected (fixated) to the functionalized surface of the sample holder and another side of the cellular body is connected to the particle.

The manipulation system of FIG. 1 may further comprise a microscope 112 with an adjustable objective 114 and a camera 116 connected with a computer 118 comprising a controller and a memory. The computer may also be programmed for tracking one or more of the particles and/or cellular bodies based on signals from the camera and/or for performing microscopy calculations and/or for performing analysis associated with super-resolution microscopy and/or video tracking, which may be sub-pixel video tracking. The computer or another controller (not shown) may be connected with other parts of the system (not shown) for controlling at least part of the microscope and/or another detector (not shown). In particular, the computer may be connected with one or more of the acoustic wave generator and the controller thereof, as shown in FIG. 1.

The system may further comprise a light source 120 for illuminating the sample using any suitable optics (not shown) to provide a desired illumination intensity and intensity pattern, e.g. plane wave illumination, Kahler illumination, etc., known per se. Here, the system light 122 emitted from the light source may be directed through the acoustic wave generator 108 to (the sample in) the sample holder 106 and sample light 124 from the sample is transmitted through the objective 114 and through an optional ocular 126 and/or further optics (not shown) to the camera 116. The objective and the camera may be integrated. In an embodiment, two or more optical detection tools, e.g. with different magnifications, may be used simultaneously for detection of sample light, e.g. using a beam splitter.

In another embodiment, not shown but discussed in detail in WO2014/200341, the system may comprise a partially reflective reflector and light emitted from the light source is directed via the reflector through the objective and through the sample, and light from the sample is reflected back into the objective, passing through the partially reflective reflector and directed into a camera via optional intervening optics. Further embodiments may be apparent to the reader.

The sample light may comprise light affected by the sample (e.g. scattered and/or absorbed) and/or light emitted by one or more portions of the sample itself e.g. by chromophores attached to the cellular bodies.

Some optical elements in the system may be at least one of partly reflective, dichroic (having a wavelength specific reflectivity, e.g. having a high reflectivity for one wavelength and high transmissivity for another wavelength), polarisation selective and otherwise suitable for the shown setup. Further optical elements e.g. lenses, prisms, polarizers, diaphragms, reflectors etc. may be provided, e.g. to configure the system 100 for specific types of microscopy.

The sample holder 102 may be formed by a single piece of material with a channel inside, e.g. glass, injection moulded polymer, etc. (not shown) or by fixing different layers of suitable materials together more or less permanently, e.g. by welding, glass bond, gluing, taping, clamping, etc., such that a holding space 106 is formed in which the fluid sample is contained, at least during the duration of an experiment.

FIG. 2A schematically depicts a flow cell for use in an acoustic force spectroscopy system according to an embodiment of the invention. The sample holder 212 may comprise a part that has a recess being, at least locally, U-shaped in cross section and a cover part to cover and close (the recess in) the U-shaped part providing an enclosed holding space 206 in cross section.

Further, the sample holder 212 may be connected to an optional fluid flow system 214 for introducing fluid into the holding space 206 of the sample holder and/or removing fluid from the holding space, e.g. for flowing fluid through the holding space (see arrows in FIG. 2A). The fluid flow system may be comprised in a manipulation and/or control system. The fluid flow system may comprise one or more of reservoirs 216, pumps, valves, and conduits 218 for introducing and/or removing one or more fluids, sequentially and/or simultaneously. The sample holder and the fluid flow system may comprise connectors, which may be arranged on any suitable location on the sample holder, for coupling/decoupling. The sample holder may further include an acoustic wave generator 222, e.g. in the form of a (at least partially transparent) piezoelectric element, connected to a controller 224.

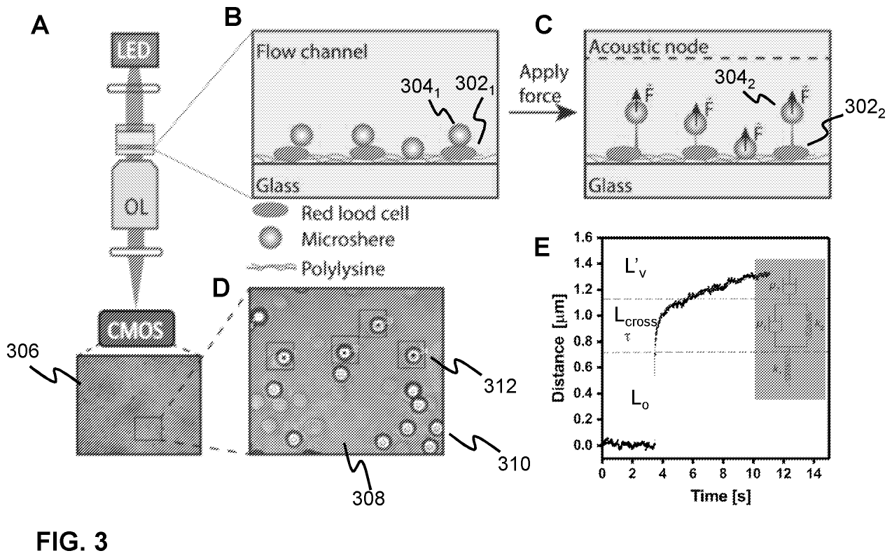

FIG. 3A-3E schematically depicts a process for probing mechanical properties of cells according to an embodiment of the invention. As shown in FIG. 3A an acoustic force spectroscopy system as e.g. described with reference to FIG. 1-2 may be used in order to examine mechanical properties of cellular bodies in the holding space of a flow cell. FIG. 3B depicts a first state of the sample under examination, including a plurality of cellular bodies 302₁, in the example Red Blood Cells (RBCs), may be attached on one side to a (bottom) surface of the flow channel of the flow cell. The surface of the flow channel may be functionalized, e.g. using Poly-L-lysine or the like, in order to fixate the RBCs to the surface. A particle 304₁, e.g. a Concanavalin A functionalized silica microsphere (approx. 6.8 micron in diameter), may be attached to each of the cellular bodies. In this state, the acoustic wave generator may be switched off so that no force is applied to the samples.

When the acoustic wave generator is switched on, a bulk acoustic standing wave will be generated at a predetermined resonant frequency in the holding space of the sample holder. This way, acoustic nodes and antinodes appear at predetermined heights in the flow cell. The acoustic field will act upon the cellular bodies and the particles, causing—in this case—a force away from the surface of the flow channel towards a node of the acoustic standing wave in the holding space.

In order to quantify the response of a body of a certain material to the acoustic field, the so-called acoustic contrast factor (Φ) is used. The acoustic contrast factor is a well-known parameter in the field as e.g. described in the article by Lenshof, A., et al, J. Acoustofluidics 5: Building microfluidic acoustic resonators. Lab Chip 12, 684 (2012). The acoustic contrast factor for a spherical object of a certain volume is given by the following expression:

$$Φ = \frac{{ρ}_{p} + 2 / 3 ( {ρ}_{p} - {ρ}_{m} )}{2 {ρ}_{p} + {ρ}_{m}} - \frac{1}{3} \frac{{ρ}_{m} {c}_{m}^{2}}{{ρ}_{p} {c}_{p}^{2}}$$

wherein ρₚ and ρₘ are the densities, and cₚ and cₘ are the speed of sound of the particle and the medium, respectively. The acoustic contrast factor Φ may be positive. In that case, a particle will experience a force in the direction of a node in an acoustic force field. If the acoustic contrast factor Φ is negative, the particle will experience a force in the direction of an antinode in an acoustic force field.

The acoustic contrast factor of a particle may be selected such that it is substantially higher (in an absolute sense) than the acoustic contrast factor of the cells, in this exemplary case, red blood cells which have an acoustic contrast factor of around 0.05 in a medium similar to water. Therefore, for a certain acoustic force field and a certain (average) particle size, the particles will experience a force, while the cellular bodies will experience a force that is negligible with respect to the force experience by the particles. This way, when the acoustic field is generated, the particles will effectively pull at the cell or push on the cell, causing the cellular body or a part thereof to deform. This is schematically shown in FIG. 3C wherein an acoustic force field will force the particles upwards, thereby pulling on the membrane of the RBCs. In this experiment, silica microspheres of relatively large dimensions (approx. 7 micron) were selected in order to achieve an effective pulling force as well as a high imaging contrast for 3-dimensional tracking compared to the RBCs. As shown in FIG. 3D, the camera captures an optical image of the sample holder comprising the RBCs. Tracking software is configured to analyse (image process) the captured images, to track selected samples (an RBC connected to a particle) and to determine displacements of the microbeads when applying a force to the samples. Known optical tracking techniques may be used as described in WO2014/200341, which is hereby incorporated by reference into this application.

In a typical experiment, a constant force is applied to the micro particles and their position is tracked over time. FIG. 3E shows an example of the viscoelastic response of a red blood cell to an applied force F of 500 pN. As shown in this figure, cells exhibit a three-phase creep response: an instantaneous elastic response I, then a retarded elastic response II, followed by viscous flow behavior III. This type of response can be described by a simple viscoelastic model consisting of springs and dashpots with stiffness's k1 and k2 and damping coefficients μ1 and μ2 (as illustrated by FIG. 3F), a four-parameter model termed Burger's viscoelastic model as described in the article by Rand et al., Biophysical Journal 4, 115-135:

$$L ( t ) = {L}_{0} + {L}_{c r o s s} \cdot [ 1 - {e}^{( - \frac{t - {t}_{0}}{τ} )} ] + {L}_{v}^{′} ( t - {t}_{0} )$$

Here, L₀ (corresponding to F/k1) is the instantaneous elastic elongation, L_{cross} (corresponding to F/k2; is the retarded elastic behavior, τ (corresponding to μ1/k2) is the characteristic time constant of the retarded elastic behavior and L′v (corresponding to F/μ2) is the long-term viscous flow. Modeling compliance by a simple combination of elastic and viscous elements, denoted by springs and dashpots, was previously used in a range of experiments, such as for lipid vesicles in fluid flow [Guevorkian et al., Biophys J 109, 2471-2479.], micro-rheological measurements on cells [Bausch et al. Biophys J 75, 2038-2049] [Bausch et al., Biophys J 76, 573-579.] and AFM studies of cell mechanics [Wu et al., Scanning 20, 389-397]. Based on this analysis, the viscoelastic behavior of cells can be described by the above-mentioned four parameters.

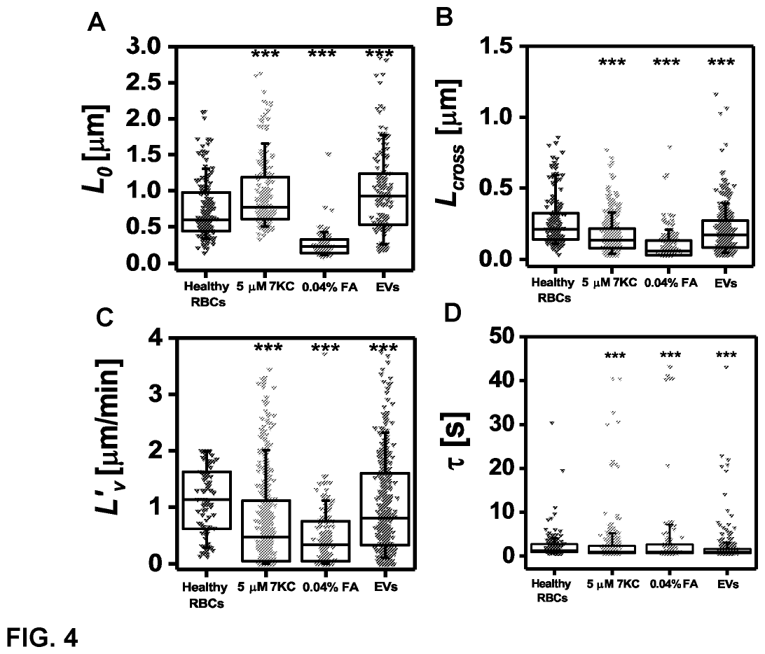

FIG. 4 depicts mechanical responses of cellular bodies measured using an acoustic force spectroscopy system according to an embodiment of the invention. In particular, FIG. 4 shows distributions of fitting parameters for red blood cell populations treated with different chemicals and or vesicles. Healthy red blood cells where compared to cells treated with 5-Cholesten-3β-ol-7-one (7KC)—a cholesterol analogue that is expected to soften the cell membrane or with formaldehyde (FA), a well-known crosslinking agent that is expected to stiffen cells. The distribution of fitting parameters L0 (A), Lcross (B), L′v (C) and τ (D) for the different treatments are shown. The difference detected was significant: in the figure samples associated with a *** reference mark correspond to a P value <0.005 compared to healthy RBCs as determined by a two-samples Kolmogorov-Smirnov test (KS test). This is a non-parametric test which quantifies the distance between the empirical distributions of two data sets, where the null hypothesis states that the two samples are drawn from the same distribution.

Besides the chemical treatments, red blood cells were also treated with red blood cell derived extracellular vesicles (EVs) by first introducing cells and microspheres into the flow cell and then exchanging the buffer solution with a buffer containing EVs derived from other RBCs (10 microliter at a concentration of 1012 particles/ml). The mechanical properties of the RBCs were probed immediately after vesicle introduction. It was expected that vesicles in the vicinity of RBC might be taken up by the RBC, and thereby change cell deformability. As RBCs do not have internal organelles, such as ER and Golgi, it was expected that uptake of vesicles might increase the surface area of the cell membrane, and thereby alter the mechanical response.

Based on the measurements it was found that such treatment indeed induces a change in RBC mechanical response: lower elastic coefficient values are obtained after vesicle treatment. A possible explanation for this increased deformability is that there is simply more available membrane to be pulled, due to incorporation of the vesicle lipids into the cell membrane. Interestingly, the long-term viscous flow (L′v) following vesicle treatment is significantly larger than for untreated RBCs similarly to the effect of 7KC, thus further supporting the explanation of increased membrane surface area.

These results show that the acoustic force spectroscopy system can be used to study the mechanical properties of cellular bodies, such as RBCs, in a multiplexed fashion, providing insights into cell mechanics. Mechanical properties of cells are essential for their function and response to the environment. Differences in stiffness can be related to several diseases, such as cancer, anemia or malaria. The embodiments in this disclosure thus provide substantial advantages over current methods for studying mechanical properties of cells, like atomic force spectroscopy, fluid flow experiments or optical tweezers. These techniques lack data throughput making it a tedious process to distinguish mechanical properties in a heterogeneous population.

When probing mechanical properties of cellular bodies using an acoustic force spectroscopy system as described in this disclosure, it is required that the particles experience a larger acoustic force than the cellular bodies. The force exerted by a given acoustic field on the particles depends on the acoustic contrast factor which quantifies the strength of the acoustic interaction of a material in a specific medium. The acoustic force further scales with the volume of the object.

The acoustic contrast factor depends on the difference in density and speed of sound of the material used compared to the medium. Cellular bodies may have an a relatively low acoustic contrast factor between −0.2 and 0.2. For example, Augustsson, P. et al. reported values between 0.03-0.11 in their article Measuring the Acoustophoretic Contrast Factor of Living Cells in Microchannels. Cell 1337-1339 (2010).

For example, red blood cells have an acoustic contrast factor of around 0.07), while materials that are commonly used for the particles have an acoustic contrast factor between 0.20 and 0.55 (e.g. the acoustic contrast factor for polystyrene is approximately 0.22, while the acoustic contract factor for silica 0.54). Hence, in order to achieve a situation in which a silica particle experiences an acoustic force that is substantially higher than the acoustic force experienced by a red blood cell it is attached to, the silica particle needs to have a size that is comparable to the size of the cellular body, e.g. around 7 micron. Therefore local probing of specific parts of a cell is not possible. Additionally, the optical tracking of the silica particle will be affected by the cell it is attached to and in more general by the cells in the optical background.

Hence, when probing mechanical properties of cells using an acoustic force spectroscopy system as described with reference to the embodiments in this application, high acoustic contrast particles may be used. Such particles enable pulling at cellular bodies with higher forces than particles of the same size but having a relatively low acoustic contrast factor. Additionally, high acoustic contrast particles allow reduction of the size of the particles thus providing higher localization accuracy.

Further, in some embodiments, the mechanics of a cell may be probed using a frequency dependent method. For example, a frequency dependent rheology method may be used. This method is a spectral method where a periodic (or sinusoidal) acoustic force signal may be applied to the particles that are attached to the cells and the responses of the particles are tracked. The response may be tracked over a range of frequencies to determine a spectral response. Smaller particles have a faster response time and thus may be advantageous for use in such spectral methods. Smaller particles enable examining the response of the cells over a broader frequency spectrum.

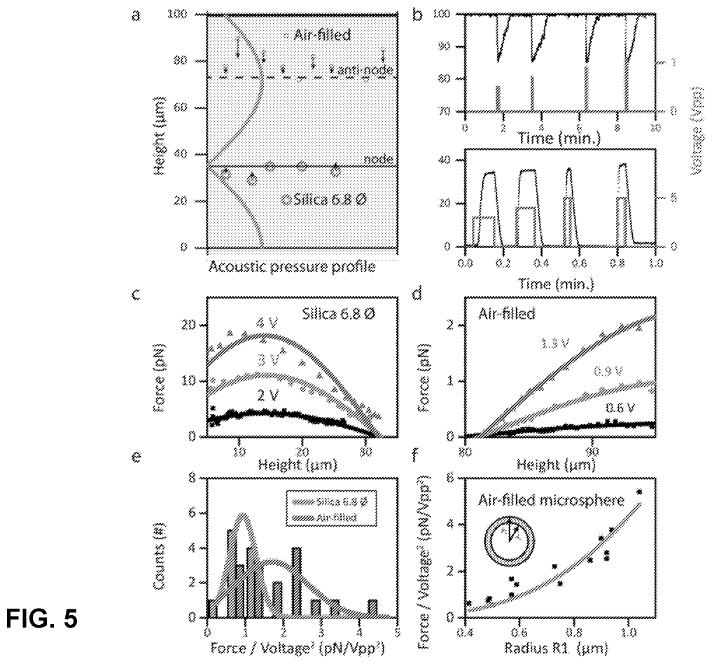

In an embodiment, high acoustic contrast factor particles may include hollow particles that are filled with a gas or air. FIG. 5A-F depict a comparison between hollow air-filled polyvinyl alcohol (PVA) particles and silica (glass) particles. In order to determine the acoustic contrast factor of the air-filled particles, the acoustic response of the air-filled microspheres is compared to the microspheres with a well-known acoustic contrast factor (silica microspheres with approx. 6.8 micron in diameter). To make a correct comparison, both microspheres are calibrated using with the same resonance frequency in the same chip. To this end, a resonance frequency is used that pushes the air-filled microspheres downwards to the acoustic anti-node and the silica microspheres upwards to the acoustic node. This is schematically shown in FIG. 5A.

Air-filled microspheres and silica microspheres can be forced in a controlled fashion to the acoustic anti-node and node, respectively as shown in FIG. 5B. The force on the microspheres at each height location is determined from the velocity with which they move from the surface to the node (or anti-node). The force profiles for different applied voltages are fitted with sine functions (FIGS. 5C and 5D) and the force/voltage² ratio is calculated for a population of silica and air-filled microspheres (FIG. 5E).

A large spread in the force/voltage² ratio for the air-filled microspheres is observed. Therefore, the upward velocity of the air-filled microspheres is used to calibrate each individual microsphere as a function of the radius. To this end, a force balance of all the forces experienced by the microsphere: the buoyance (Fb), gravitation (Fg) and the stokes drag force (F_{Stokes}) may be determined and solved for the velocity:

$${F}_{b} + {F}_{g} + {F}_{Stokes} = 0$$

$${F}_{g} = Vpg = \frac{4}{3} π g ( ( {R}_{2}^{3} - {R}_{1}^{3} ) {ρ}_{PVA} + {R}_{1}^{3} {ρ}_{air} )$$

$${F}_{Stokes} = - 6 π η {R}_{2} v$$

$$v = \frac{{2}_{g}}{9 η {R}_{2}} ( ( {R}_{2}^{3} - {( {R}_{2} - d )}^{3} {ρ}_{PVA} + {( {R}_{2} - d )}^{3} {ρ}_{air} - {R}_{2}^{3} {ρ}_{water} )$$

here V represents the volume of the microsphere, ρ the density, R₁ and R₂ the inner and the outer radius of the microsphere, respectively, η the viscosity of the medium and d the shell thickness (R₂−R₁=300 nm).

Since the acoustic force scales with the volume of the particle, the force/V² ratio is plotted against the inner radius and fitted with a third power function (FIG. 5F). Here, V is the voltage applied over the piezo element used to control the acoustic force, as expected, the force scales quadratically with the applied voltage. When extrapolating this function to the radius of the silica microspheres (approx. 3.4 micron), it was found that the air-filled microspheres experience 170±14 fold higher force than the silica microspheres, but in the opposite direction. As a result, it was found that the acoustic contrast factor is −170·0.54=−92±7.

Thus, compared to polystyrene microspheres (a commonly used particle material) the increase in force is about 400-fold, which means at least 7 times smaller microspheres can be used and still exert the same force on the microspheres. This way, smaller microspheres or even sub-micron spheres (nanospheres) can be used to exert forces on a cell. Furthermore, microspheres can be selected that are (substantially) smaller than the typical dimensions of a cell to probe specific parts of the cell instead of the mechanical response of the whole cell. The use of particles smaller than the cells which are probed also may have an advantage for throughput: using smaller particles, more particles can be tracked within the same field-of-view.

Hence, high acoustic contrast particles referred to in the embodiments of this disclosure may include hollow organic particles, e.g. hollow polymer-based particles as described with reference to FIGS. 5A-5F, and inorganic hollow particles, e.g. hollow glass particles.

In an embodiment, the particles may include hollow particles, which may be filled with a gas, a gas mixture (including air) or a liquid. The hollow particles may include hollow inorganic particles, e.g. oxide-based, e.g., silicon oxide-based hollow particles, glass-based hollow particles or ceramic-based hollow particles. Alternatively, the hollow particles may include hollow organic particles. Such hollow particles may include polymer-based hollow particles, e.g. polyvinyl alcohol (PVA) based hollow particles.

In yet another embodiment, the hollow particles may have an acoustic contrast factor (in a water-type fluid medium) that is larger than 0.5 or smaller than −0.5. In another embodiment, the hollow particles may have an acoustic contrast factor (in a water-type fluid medium) that is larger than 0.6 or smaller than −0.6.

Further, in an embodiment, hollow particles with a low compressibility (e.g. a ‘hard’ shell) may be selected. Low-compressibility particles are desired because highly compressible particles as e.g. used as ultrasound contrast agents generate a strong local acoustic field around themselves when placed in an acoustic field. This extra acoustic field distorts the force applied to the cell to which it is attached to and/or influences other nearby particles. Hence, preferably the hollow particles may have a shell of a material with a high Young's modulus, preferably a Young's modulus selected between 1 and 1000 GPa. For example, in an embodiment, the shell may include a glass material having a Young's modulus between 50-90 GPa. In another embodiment, the shell may include a polyvinyl alcohol material having a Young's modulus between 1-10 GPa. In yet a further embodiment, the shell may include a ceramic material having a Young's modulus of more than 50 GPa.

The air-filled microspheres further provide the advantage of high optical contrast. As shown in FIG. 3D, cells are imaged using an optical microscope. For accurate measurements, the microspheres that are placed on top of the cells, need to be tracked in three dimensions, in particular in the direction of the z-axis (the direction perpendicular to the surface of the sample holder). Because z-tracking relies on precise analysis of radial ring patterns that appear around a particle, the image of the cells can interfere with the ring patterns.

Additionally, for accurate optical tracking against a background of cells, the particles need to have an optical contrast that is higher, preferably substantial higher, than the optical contrast of the cells. The optical contrast depends on the difference between the refractive index between the object and the medium. Water (the medium) has a refractive index of 1.33 and glass has 1.5 (13% difference), while red blood cells have a refractive index of −1.4 (5% difference). Hence, the microbeads have a higher optical contrast compared to the red blood cells, thus optical tracking of the microbeads in the z-direction is possible. Nevertheless, improvements in the optical contrast are desirable, especially since the particles need to be tracked against a cellular background.

The optical contrast may be optimized by choosing particles with a sufficiently different refractive index. This may be realized selecting a material with a high refractive index, or at least a high real part of the refractive index in the visible range. For example, for diamonds, the real part of the refractive index is 2.4-2.5 in the visible range, thus providing a refractive index difference between 80% and 88%.

Similarly, this may be realized selecting a material with a low refractive index, or at least a low real part of the refractive index in the visible range. For example, air in air-filled hollow particles has a refractive index of 1 (25% difference), thus providing a substantial higher difference in refractive index when compared with glass (silica) particles. The air-filled hollow particles thus not only provide a high acoustic contrast factor but also high optical visibility when compared with solid silica particles.

In a further embodiment, solid particles of a high acoustic contrast factor material may be selected. For example, in an embodiment, diamond particles may be selected. Diamond particles will have an acoustic contrast factor that is approximately three times higher than polystyrene particles.

The (average) size of the particles (microparticles and nanoparticles) and the contrast factor may be selected on the basis of a particular application. For example, for locally probing mechanical properties of a cellular body, the size of a particle may be substantially smaller than the dimensions of the cellular body. For example, in an embodiment, for probing local mechanical parameters, the size of the particles may be selected to be 20% of the cell size or smaller. In another embodiment, for probing global parameters the size of the particles may be selected to be 50% of the cell size or larger.

Taken into account application specific conditions (e.g. measuring global or local mechanical parameters, measuring cells with a relatively high acoustic contrast factor and/or optical refraction index, applying a relatively large force, etc.), the (average) size of the particle may be selected 0.2 and 20 micron wherein—depending on the cell type—the size of the cells may vary between 3 and 100 micron.

The surface of the particles described in this disclosure may be functionalized in order to adhere to a cell and/or a specific part of a cell. Known materials may be used to functionalize the surface of the particles. For example, in an embodiment, a particle is functionalized using one or more primers comprising one or more interaction moieties for adhesion to at least part of the cellular body, preferably an interaction moiety including at least one of: viruses, viral particles, antibodies, peptides, biological tissue factors, biological tissue portions, antigens, proteins, ligands, lipid (bi)layers, fibronectin, cellulose, nucleic acids, RNA, small molecules, allosteric modulators, (bacterial) biofilms, and specific atomic or molecular surface portions (e.g. a gold surface).

In an embodiment, hollow particles PVA particles may be functionalized using antibodies in order to adhere particles to certain cell types, such as cancer cells as e.g. described by Faridi et al, in their article MicroBubble Activated acoustic cell sorting Biomed Microdevices. 2017 June; 19(2):23.

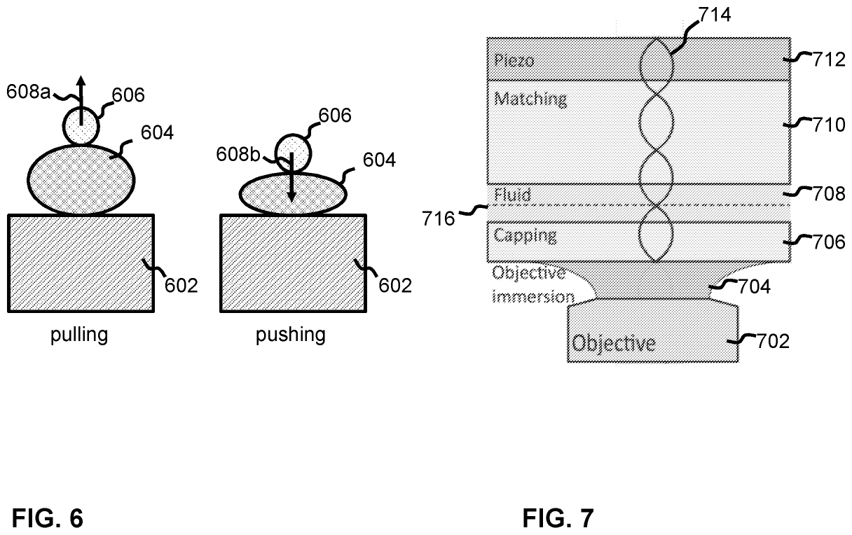

FIG. 6 schematically depicts a process for probing mechanical properties of cells according to another embodiment of the invention. In this embodiment, forces can be applied in two directions by changing the applied resonance frequency. A sample holder (a chip) has a specific configuration of layers leading to a set of resonance frequencies associated with a specific force profile in z direction of the sample holder. As shown in FIG. 6, cell 604 may be bound to a substrate 602 and a particle 606 may be bound to the cell. A force indicated by an arrow can be applied to the particle in a direction away from the substrate 608a which means the particle pulls on the cell. Alternatively, it can be applied in a direction towards the substrate 608b which means that the particle pushes on the cell. Pushing and pulling can probe different aspects of the cell mechanics: while pulling probes both the mechanics of the membrane and the cytoskeleton, pushing applies direct load on the cytoskeleton of the cell, which dominates the mechanical properties of the membrane of the cell.

FIG. 7 schematically depicts a cross-section of part of a sample holder for an acoustic force spectroscopy system according to an embodiment of the invention. The sample holder may include an objective 702 that is positioned underneath part of a sample holder (a chip) wherein the sample holder may comprise a capping layer 706, a matching layer 710, a fluid 708 contained in the holding space formed by the capping and the matching layer, and a piezo element 712. By applying an AC voltage V to the piezo element at the appropriate frequency, a resonant bulk acoustic standing wave can be generated in the sample holder which optionally has a node 716 in the fluid layer. Particles that have a positive acoustic contrast factor with respect to the fluid medium will be attracted to the acoustic node. In an embodiment, an immersion fluid 704 between the objective and the capping layer may be used to improve the optical NA of the imaging system.

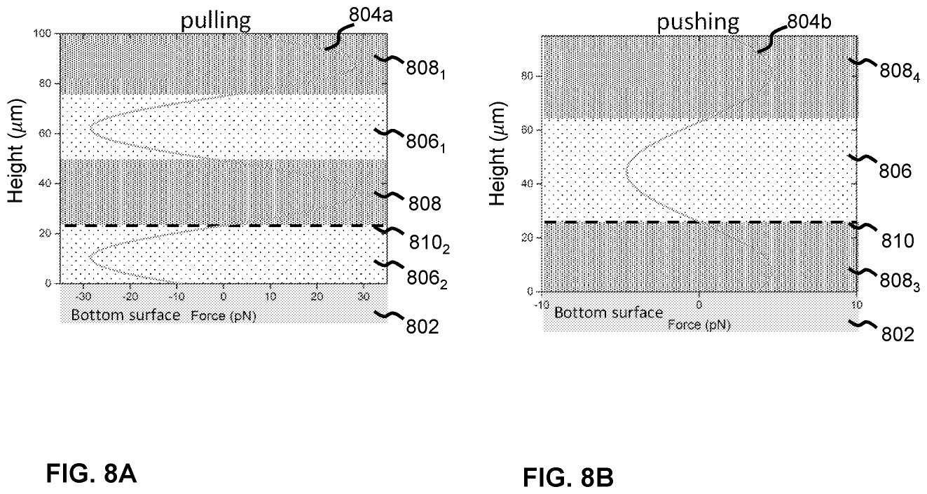

FIG. 8 schematically depicts acoustic resonant modes of part of a sample holder for an acoustic force spectroscopy system according to an embodiment of the invention. In particular, FIGS. 8A and 8B depict two (non-limiting) examples of force profiles created by the acoustic standing wave over the height of the fluid channel of the sample holder. Different force profiles may be shaped in the sample holder by tuning the frequency of the applied wave or by changing the material properties and dimensions of elements that form the sample holder, including the piezo, the matching layer, the fluid layer and/or the capping layer (see FIG. 7).

The figure shows a part of the capping layer 802, a part of the fluid layer with a resonant standing wave 804a,804b. As shown in these figures, the fluid layer contains regions of negative force 806 and positive force 808 as would be experienced by a particle with a positive acoustic contrast. For example, in the first example of FIG. 8A, the fluid layer includes two regions of negative force 806_{1,2} and two regions of positive force 808_{1,2}. Similarly, the second example of FIG. 8B depicts two regions of positive force 808_{3,4} and one region of a negative force 806₃. The first acoustic node as seen from the bottom surface 810 is also indicated. In the regions of negative force a particle of positive acoustic contrast would experience a force away from the bottom surface. In the regions of positive force a particle of positive acoustic.

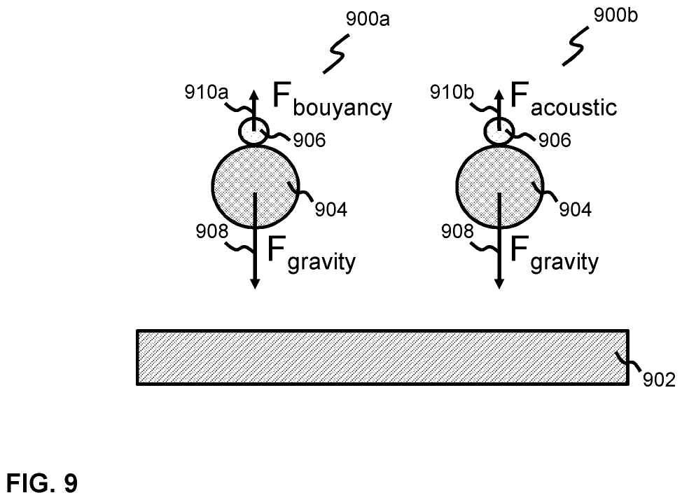

FIG. 9 depicts a method for preparing cells for a mechanical probing process according to an embodiment of the invention. The method provides an increased yield of cell-particle constructs for a mechanical probing process.

In order to measure many cells in parallel and gather enough data for proper statistical analysis, it may be advantageous to create a sample substrate that has many cells in the field-of-view of the microscope wherein a particle is attached to the top of each cell. In some embodiments, it may be advantageous that the particle is nicely centred on top of the cell. FIG. 9 depicts the preparation of a sample substrate wherein cells are positioned on the surface of the sample holder 902 having a particle positioned on top of the cell. The preparation of the sample substrate may include the steps of: mixing particles 906 with cells 904 and flushing them into the sample chamber. During this process, a particle that has a functionalized surface may adhere to a surface of a cell. Thereafter, the gravitational force F_{gravity} 908 may cause the cells to sink to the bottom surface where they can attach to the substrate.

In an embodiment 900b, particles may have a density higher than the density of the medium. In that case, a gentle acoustic force 910b may be applied that acts on the particle, while the gravity force acts on the cell. These forces will align the cell—particle construct with the axis perpendicular to the surface of the sample holder. In the aligned position, the particle is on top of the cell as depicted in the figure. The acoustic force generator may be controlled to generate an acoustic force F_{acoustic} onto the particles that is smaller than the gravitational force on the particles. The acoustic force may be slowly lowered while the particles sink to the surface as required. This way, cell particle constructs may be deposited in a controllable way onto the surface of the holding space of the sample holder wherein the particles are positioned on top of the cellular bodies.

In an embodiment 900a, the particles may have a density lower than the density of the medium, e.g. hollow gas or air filled particles. In that case, the particles may generate a buoyancy force F_{bouyancy} 910a. The buoyancy force that acts on the particle and the gravitational force that acts on the cell may cause the cell—particle construct to align with the axis perpendicular to the surface of the sample holder. In the aligned position, the particle is on top of the cell as depicted in the figure. The gravity force will gently pull the cells towards the surface of the sample holder, while the buoyancy force keeps the particle on top of the cell. This way, cells are deposited in a controllable way onto the surface of the holding space of the sample holder wherein the particles are positioned on top of the cellular bodies. This way, a high trough put sample substrate is realized having many cell—particle constructs that are suitable for use in mechanical probing experiments.

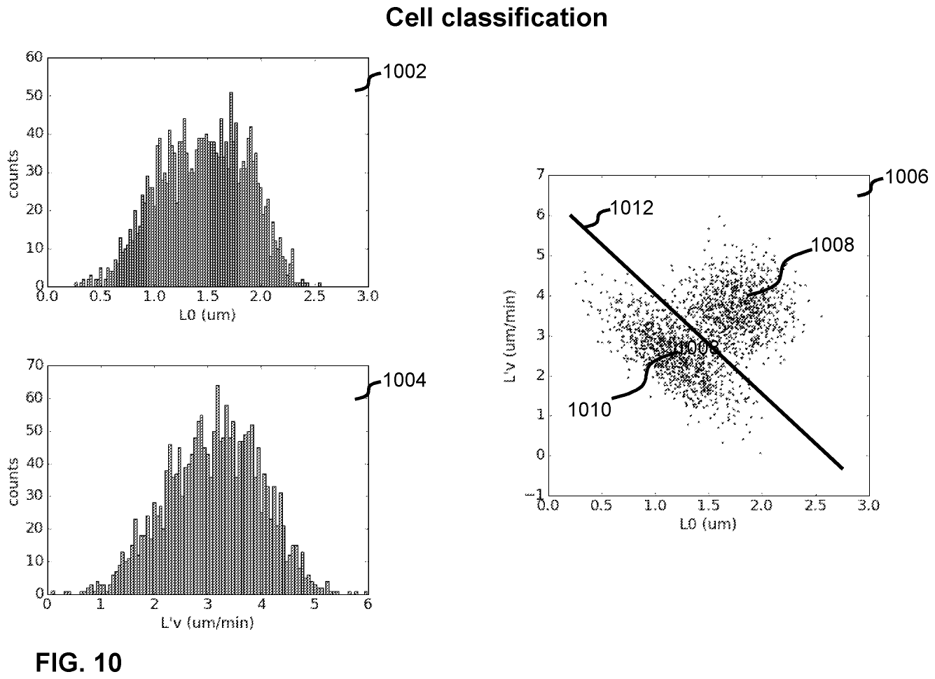

FIG. 10 schematically depicts a process of classifying cells on the basis of mechanical parameters according to an embodiment of the invention. In particular, FIG. 10 illustrates a method for classification of different cell populations based on multi-dimensional analysis. Graphs 1002 and 1004 represent histograms of mechanical parameters L0 and L′v of a simulated dataset based on two cell populations where these parameters are differently correlated for the two populations. Based on the histograms, it is not obvious that this dataset contains to different cell populations. When the data is plotted on a 2D graph 2006 with the two parameters on the two axes, a clear separation between the two cell populations, a first cell population 1008 associated with one or more first cell parameters and a second cell population 1010 associated with one or more cell parameters becomes apparent. This way, cells can be classified (assigned to either one of the populations) based on whether the data points are located above or below a separation line 1012. This method can obviously be extended to any number of dimensions.

Hence, using for example the burgers model (as is explained above with reference to FIG. 3E) a number of fit parameters can be obtained using the mechanical probing techniques as described with reference to the embodiments of this disclosure. Analyzing the fit parameters in 2D, 3D or even 4D space allows classifying differences in responses between groups of cells. Such classification scheme may be used to distinguish between healthy and diseased cells.

The disclosure is not restricted to the above described embodiments which can be varied in a number of ways within the scope of the claims as explained supra. Elements and aspects discussed for or in relation with a particular embodiment of the method or system may be suitably combined with elements and aspects of other embodiments of the system or method, unless explicitly stated otherwise.

1. A method for probing mechanical properties of cellular bodies, the method comprising steps of:
providing a plurality of particles and cellular bodies in a fluid medium contained in a holding space of a sample holder and fixating the cellular bodies to a surface of the holding space, wherein each of the plurality of particles is attached to one of the cellular bodies;
generating a resonant bulk acoustic wave in the holding space, the resonant bulk acoustic wave exerting an acoustic force on each of the plurality of particles in a direction away from the surface of the holding space or in a direction towards the surface of the holding space, each of the plurality of particles having an acoustic contrast factor and a size, the acoustic contrast factor and the size being selected such that the force exerted on one said particle is larger than the force exerted on the cellular body to which the one said particle is attached; and,
for at least part of the particles which are attached to a cellular body, measuring, one or more displacements of one said particle in response to the exertion of the force on the one said particle, the measured one or more displacements being associated with at least one mechanical property of the cellular body.

2. The method according to claim 1 wherein the particles have an acoustic contrast factor that is larger than 0.5 or smaller than −0.5.

3. The method according to claim 1, wherein the particles include hollow particles, which hollow articles may be filled with a gas, a gas mixture or a liquid.

4. The method according to claim 3 wherein the hollow particles have a low compressibility, the hollow particles comprising a shell material having a Young's modulus selected between 1 and 1000 GPa.

5. The method according to claim 3, wherein the hollow particles have a shell thickness between 0.1 and 5 microns.

6. The method according to claim 1, wherein a material of one said particle has an optical refractive index that differs at least 80% from a refractive index of the cellular body to which the particle is attached.

7. The method according to claim 1, wherein the size of the particles is selected to be 20% of the size of the cellular body or smaller; and/or, wherein the size of the particle is selected to be between 0.2 and 20 micron and wherein the size of the cellular body is selected to be between 1 and 100 micron.

8. The method according to claim 1, wherein at a first acoustic resonant frequency the force exerted on one said particle is in a direction away from the surface of the flow cell and wherein at a second acoustic resonant frequency the force exerted on the one said particle is in a direction towards the surface of the holding space of the sample holder.

9. The method according to claim 1, wherein at least a portion of the particles and/or at least a portion of the surface of the holding space is/are functionalized using one or more primers comprising one or more interaction moieties type(s) for adhesion to at least part of the cellular body, wherein said interaction moieties type(s) include at least one type selected from the group consisting of: viruses, virus particles, antibodies, peptides, biological tissue factors, biological tissue portions, antigens, proteins, ligands, lipid layers, fibronectin, cellulose, nucleic acids, RNA, small molecules, allosteric modulators, biofilms, and specific atomic or molecular surface portions.

10. The method according to claim 1 further comprising steps of:
providing a plurality of the cellular bodies in the medium of the holding space of the sample holder, one said particle being attached to each of the plurality of the cellular bodies;
controlling the resonant bulk acoustic wave in the sample holder in order to exert the force on the particles, the acoustic force being selected to be smaller than a gravitational force that pulls cellular bodies towards the surface of the holding space;
the gravitational force depositing the cellular bodies onto the surface of the holding space, wherein the acoustic force acting on one said particle attached to one said cellular body ensures that the on said cellular body will land onto the surface of the holding space with the one said particle on top of the one said cellular body.

11. The method according to my claim 1, further comprising steps of:
providing a plurality of the cellular bodies in the medium of the holding space of the sample holder, one said particle being attached to each of the plurality of cellular bodies, wherein the particles have a density lower than tea density of the medium of the holding space;
a gravitational force depositing the cellular bodies onto the surface of the holding space, wherein a buoyancy force of one said particle attached to one said cellular body ensures that the one said cellular body will land onto the surface of the holding space with the one said particle on top of the one said cellular body.

12. The method according to claim 1, further comprising steps of:
providing a plurality of the cellular bodies and a plurality of the particles in the medium of the holding space of the sample holder;
controlling the resonant bulk acoustic wave in the sample holder in order to exert the force on the particles, the acoustic force being selected to be smaller than a gravitational force that pulls the plurality of the cellular bodies towards the surface of the holding space and the force being selected such that the particles are trapped in a node or an antinode of the resonant bulk acoustic wave, the gravitational force depositing the cellular bodies onto the surface of the holding space;
controlling another resonant bulk acoustic wave in the sample holder to release the particles from the node or antinode, the gravitational force on the particles depositing the particles onto the cellular bodies for attaching the particles to the cellular bodies.

13. The method of claim 1 further comprising the steps of:
for at least part of the particles attached to the cellular bodies, measuring displacements of said at least part of the particles as a function of time; and
classifying each of the cellular bodies on the basis of the measured displacements.

14. A system for probing mechanical properties of cellular bodies, the system comprising:
a sample holder comprising a holding space for holding a sample, the sample comprising a plurality of particles and cellular bodies in a fluid medium contained in the holding space of the sample holder, the cellular bodies being fixated to a surface of the holding space, wherein each of the plurality of particles is attached to one of the cellular bodies;
an acoustic wave generator connectable or connected with the sample holder to generate an acoustic wave in the holding space for exerting a force on the sample;
a detector for detecting a response of the sample to the acoustic wave; and,
a controller module for controlling the acoustic wave generator and the detector, the controller module including;
a computer readable storage medium having computer readable program code embodied therewith, and a processor coupled to the computer readable storage medium, wherein responsive to executing the computer readable program code, the processor is configured to perform executable operations comprising:
controlling the acoustic wave generator to generate a resonant bulk acoustic wave in the holding space, the resonant bulk acoustic wave exerting an acoustic force on each of the plurality of particles in a direction away from the surface of the holding space or in a direction towards the surface of the holding space, each of the plurality of particles having an acoustic contrast factor and a size, the acoustic contrast factor and the size being selected such that the force exerted on a particle is larger than the force exerted on the cellular body to which the particle is attached; and
controlling the detector to measure for at least part of the particles attached to a cellular body, one or more displacements of one said particle in response to the exertion of the force on the one said particle, the one or more measured displacements being associated with at least one of the mechanical properties of the cellular body.

15. The system according to claim 14, the executable operations further comprising:
computing at least one of the mechanical properties of the cellular body based on the one or more measured displacements.

16. The method according to claim 1, wherein the particles are microparticles or nanoparticles and the measuring step is carried out by an optical detector.

17. The method according to claim 2 wherein the particles have an acoustic contrast factor that is larger than 0.6 or smaller than −0.6.

18. The method according to claim 3, wherein the particles include hollow inorganic particles and/or the hollow particles including hollow organic particles.

19. The method according to claim 4, wherein the hollow particles have a low compressibility, the hollow particles comprising a shell material having a Young's modulus selected between 50 to 90 GPa.

20. The method according to claim 6, wherein a material of one said particle has an optical refractive index that differs at least 20-25% from a refractive index of the cellular body to which the particle is attached.
